(19) United States
(12) Patent Application Publication
(10) Pub. No.: US 2004/0109882 A1
(43) Pub. Date: Jun. 10, 2004

(54) Use of flavones, flavanones and flavonoids for protecting ascorbic acid and/or ascorbyl compounds from oxidation

(75) Inventors: Uwe Schonrock, Nahe (DE); Inge Kruse, Hamburg (DE)

Correspondence Address: Norris, McLaughlin & Marcus P.A., 30th Floor, 220 East 42nd Street, New York, NY, 10017, US

(73) Assignee: Beiersdorf AG, Hamburg (DE)

(21) Appl. No.: 10/725,229

(22) Filed: Dec. 1, 2003

Related U.S. Application Data
(62) Division of application No. 09/243,568, filed on Feb. 3, 1999, now abandoned.

(30) Foreign Application Priority Data
Feb. 24, 1998 (DE) 198 07 774.2

(51) Int. Cl. A61K 31/7048; A61K 31/375; A61K 31/353

(52) U.S. Cl. 424/401; 514/27; 514/456; 514/474

(57) ABSTRACT

Use of at least one active ingredient chosen from the group consisting of flavones, flavanones and flavonoids for protecting at least one active ingredient chosen from the group consisting of ascorbic acid and ascorbyl compounds from oxidation.

The present invention relates to the use of flavones, flavanones and flavonoids for protecting ascorbic acid and/or ascorbyl compounds in general from oxidation, in particular in cosmetic and dermatological preparations. The present invention preferably relates to cosmetic preparations with an effective protection against harmful oxidation processes in the skin, but also to the protection of cosmetic preparations themselves and to the protection of the constituents of cosmetic preparations against harmful oxidation processes.

The present invention further relates to antioxidative active ingredient combinations, preferably those used in skincare cosmetic or dermatological preparations. In particular, the invention also relates to cosmetic and dermatological preparations comprising such antioxidants.

In a preferred embodiment, the present invention relates to cosmetic and dermatological preparations for the prophylaxis and treatment of cosmetic or dermatological skin changes, such as, for example, skin ageing, in particular skin ageing caused by oxidative processes.

The present invention further relates to active ingredients and preparations comprising such active ingredients for the cosmetic and dermatological treatment or prophylaxis of erythematous, inflammatory, allergic or autoimmune-reactive symptoms, in particular dermatoses.

The present invention further relates to active ingredient combinations and preparations used for the prophylaxis and treatment of light-sensitive skin, in particular of photodermatoses.

The damaging effect of the ultraviolet part of solar radiation on the skin is generally known. While rays having a wavelength of less than 290 nm (the UVC region) are absorbed by the ozone layer in the earth's atmosphere, rays in the region between 290 nm and 320 nm, the UVB region, cause erythema, simple sunburn or even burns of varying severity.

The erythema activity maximum of sunlight is stated as the narrower region around 308 nm.

Numerous compounds are known for protecting against UVB radiation; these are usually derivatives of 3-benzylidenecamphor, 4-aminobenzoic acid, cinnamic acid, salicylic acid, benzophenone and also 2-phenylbenzimidazole.

For the region between about 320 nm and about 400 nm, the UVA region, it is also important to have available filter substances, since the rays of that region can also cause reactions in the case of light-sensitive skin. It has been found that UVA radiation leads to damage of the elastic and collagenic fibres of connective tissue, causing premature ageing of the skin, and that it is to be regarded as a cause of numerous phototoxic and photoallergic reactions. The damaging effect of UVB radiation can be intensified by UVA radiation.

To protect against the rays of the UVA region, therefore, certain derivatives of dibenzoylmethane are used, the photostability of which is insufficient (Int. J. Cosm. Science 10, 53 (1988)).

However, UV radiation can also lead to photochemical reactions, in which case the photochemical reaction products intervene in the skin's metabolism.

Such photochemical reaction products are predominantly free-radical compounds, for example hydroxyl radicals. Undefined free-radical photoproducts which are formed in the skin itself can also display uncontrolled secondary reactions because of their high reactivity. However, singlet oxygen, a non-radical excited state of the oxygen molecule, can also be formed during UV irradiation, as can short-lived epoxides and many others. Singlet oxygen, for example, differs from the normal triplet oxygen (free-radical ground state) by its increased reactivity. However, excited, reactive (free-radical) triplet states of the oxygen molecule also exist.

UV radiation is also a type of ionizing radiation. There is therefore the risk that UV exposure may also produce ionic species, which then, for their part, are capable of oxidative intervention in the biochemical processes.

All of these effects subject the cell to oxidative stress, which is taken to mean the burdening of the living cell as a result of a concentration in cell-damaging oxidized compounds such as, for example, lipid hydroperoxides, hydrogen peroxide or reactive oxygen compounds such as singlet oxygen, hydroxyl, and hyperoxide anions. Oxidative stress can be induced, for example, by the effect of radiation, xenobiotics, heavy metal ions or other causes. It plays a role in the emergence of a number of diseases.

The body does have has a limited number of defence mechanisms against oxidative stress. For example, with the help of various enzyme activities [e.g. superoxide dismutase, peroxidases such as glutathione peroxidase (see glutathione), catalase, caeruloplasmin], it is possible to prevent the occurrence of reactive free radicals. This, however, is often insufficient and must be supported, be it on a cosmetic or medical level.

In order to avoid oxidative stress, it is possible to incorporate additional antioxidants and/or free-radical scavengers into the cosmetic or dermatological formulations.

It has already been proposed to use vitamin E, a substance with a known antioxidative effect in light protection formulations, but the effect achieved falls a long way short of expectations here as well.

The object of the invention was therefore also to provide cosmetic, dermatological and pharmaceutical active ingredients and preparations and light protection formulations which are used for the prophylaxis and treatment of light-sensitive skin, in particular photodermatoses, preferably PLD.

Other names for polymorphous light dermatosis are PLD, PLE, Mallorca acne and a large number of other names, as given in the literature (e.g. A. Voelckel et al, Zentralblatt Haut-und Geschlechtskrankheiten (1989), 156, p.2).

Erythematous skin symptoms also occur as accompanying symptoms in certain skin diseases or irregularities. For example, the typical skin rash symptom of acne is generally red to a greater or lesser extent.

Antioxidants are mainly used as substances which protect against spoilage of the preparations in which they are present. Nevertheless, it is known that in human and animal skin as well undesired oxidation processes may occur. Such processes play an important role in skin ageing.

The essay “Skin Diseases Associated with Oxidative Injury” in “Oxidative Stress in Dermatology”, p. 323 ff. (Marcel Decker Inc., New York, Basel, Hong Kong, Editor: Jürgen Fuchs, Frankfurt, and Lester Packer, Berkeley/Calif.) discusses oxidative skin damage and its more obvious causes.

If the aim is to permanently colour human hair, only oxidizing hair colouring methods are suitable in practice. During oxidative hair colouring, dye chromophores form as a result of the reaction of precursors (phenols, aminophenols, more rarely also diamines) and bases (in most cases p-phenylenediamine) with the oxidizing agent, in most cases hydrogen peroxide. Hydrogen peroxide concentrations around 6% are normally used.

It is usually assumed that in addition to the colouring action, a bleaching action as a result of the hydrogen peroxide also takes place. In oxidatively coloured human hair, as is the case for bleached hair, it is possible to detect microscopic holes at sites where melanin granules were present.

The fact is that the oxidizing agent hydrogen peroxide reacts not only with the dye precursors, but also with the hair substance, and in some instances can cause hair damage.

Antioxidants are substances which prevent oxidation processes or which prevent the autoxidation of fats containing unsaturated compounds. Antioxidahts, which are also used in the fields of cosmetics and pharmaceuticals, are, for example, α-tocopherol, in particular in the form of the α-tocopheryl acetate, sesamol, bile acid derivative, butylated hydroxyanisole and butylated hydroxytoluene.

Also for reasons of preventing such reactions, it is possible to additionally incorporate antioxidants and/or free-radical scavengers into cosmetic formulations.

Antioxidants are chemical substances having a variety of structures which inhibit or prevent undesired changes in the substances to be protected which are caused by the effects of oxygen and other oxidative processes. Antioxidants are used in particular for protecting organic products, namely fats and oils, but also active ingredients and additives, such as, for example, aroma substances and the like. Examples of effective antioxidants are phenols substituted by sterically hindering groups, hydroquinones, pyrocatechols and aromatic amines and the metal complexes thereof. Substances suitable for fats, foods and, in particular, also cosmetic and dermatological preparations are tocopherol, butylated hydroxyanisole (BHA), butylated hydroxytoluene (BHT), octyl gallate and dodecyl gallate and also ascorbic, lactic, citric and tartaric acids and salts thereof.

Although a number of antioxidants and free-radical scavengers are known, for example U.S. Pat. Nos. 4,144,325 and 4,248,861 and a large number of other documents have already proposed using vitamin E, a substance with a known antioxidative action in light protection formulations, the effect achieved nevertheless remains a long way behind that desired in this case as well.

Excellent antioxidants per se are chosen from the group of ascorbic acid and ascorbyl compounds.

L-Ascorbic acid {(R)-5-[(S)-1,2-dihydroxyethyl]-3,4-dihydroxy-5-H-furan-2-one, vitamin C} is characterized by the structural formula

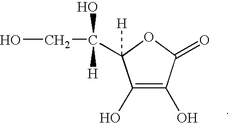

It is freely soluble in water, readily soluble in alcohol, insoluble in ethers, petroleum ethers, chloroform, benzene and in fats and fatty oils. Ascorbic acid is an enediol and, as a reductone, has a strongly reducing effect. Ascorbic acid is heat-sensitive and is decomposed, in particular in the presence of traces of heavy metal and in an alkaline medium, by light and atmospheric oxygen, whilst in a pure, dry state it is relatively resistant towards light, air and heat.

In cosmetic and dermatological preparations, ascorbyl compounds are often used instead of ascorbic acid, preferably ascorbyl esters of fatty acids, particularly preferably ascorbyl palmitate, since the sensitivity of these compounds to an oxidative effect is much less compared with ascorbic acid, and most of these compounds are more soluble in oil, which may offer pharmaceutical advantages.

Ascorbyl compounds in the narrower sense are, in particular, the ascorbyl esters of the general structure

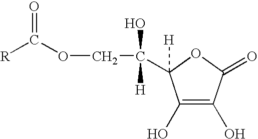

where R may be a branched or unbranched alkyl radical having up to 25 carbon atoms.

One object of the present invention was to overcome the disadvantages of the prior art. In particular, the aim was to provide active ingredients or preparations comprising such active ingredients, the use of which is at least able to reduce, if not completely prevent, damage to skin and/or hair by oxidative effects.

A further object of the present invention was to provide cosmetic preparations which, before or after treatment of the hair with hair colouring preparations, even those containing strong oxidizing agents such as, for example, hydrogen peroxide, counter the damaging oxidation effect thereof.

In particular, the aim was to provide active ingredients and preparations comprising such active ingredients for the cosmetic and dermatological treatment and/or prophylaxis of erythematous, inflammatory, allergic or autoimmune-reactive symptoms, in particular dermatoses, but also the symptom of “stinging”.

A particular objective was to find ways of protecting ascorbyl compounds, in particular vitamin C and vitamin C esters from harmful oxidative effects, preferably in cosmetic or dermatological preparations.

The use of flavones and flavonoids in cosmetics and dermatology is known per se. For example, DE-A 44 44 238 describes combinations of cinnamic acid derivatives and flavone glycosides, for example α-glycosylrutin, as antioxidants and as active ingredients against other indications.

It was therefore surprising and could not have been foreseen by the person skilled in the art that the use of at least one active ingredient chosen from the group consisting of flavones, flavanones and flavonoids for protecting at least one active ingredient chosen from the group consisting of ascorbic acid and ascorbyl compounds from oxidation, in particular for protecting against oxidation in cosmetic or dermatological preparations, overcomes the disadvantages of the prior art.

Flavone and its derivatives (often also collectively called “flavones”) are characterized by the following basic structure (substitution positions are given):

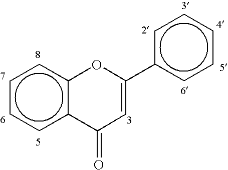

Some of the more important flavones, which can also be found in living nature, are given in the table below:

OH substitution positions

| | 3 | 5 | 7 | 8 | 2′ | 3′ | 4′ | 5′ |
|---|---|---|---|---|---|---|---|---|
| Flavone | − | − | − | − | − | − | − | − |
| Flavonol | + | − | − | − | − | − | − | − |
| Chrysin | − | + | + | − | − | − | − | − |
| Galangin | + | + | + | − | − | − | − | − |
| Apigenin | − | + | + | − | − | − | + | − |
| Fisetin | + | − | + | − | − | + | + | − |
| Luteolin | − | + | + | − | − | + | + | − |
| Kaempferol | + | + | + | − | − | − | + | − |
| Quercetin | + | + | + | − | − | + | + | − |
| Morin | + | + | + | − | + | − | + | − |
| Robinetin | + | − | + | − | − | + | + | + |
| Gossypetin | + | + | + | + | − | + | + | − |
| Myricetin | + | + | + | − | − | + | + | + |

In nature, flavones are usually in glycosylated form.

Flavonoids are glycosides of flavones, of flavanones, the basic skeleton of which is characterized by the following structure:

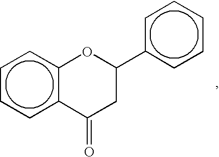

of 3-hydroxyflavones (flavonols), the basic skeleton of which is characterized by the following structure:

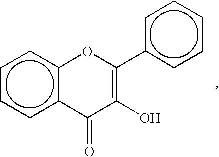

of aurones, the basic skeleton of which is characterized by the following structure:

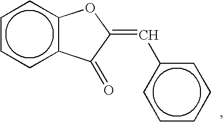

and also of isoflavones, the basic skeleton of which is characterized by the following structure:

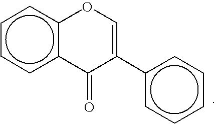

According to the invention, the flavonoids are preferably chosen from the group of substances having the generic structural formula

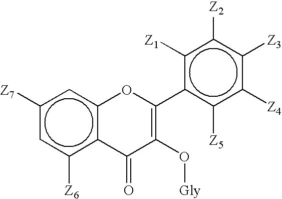

where Z₁-Z₇ independently of one another are chosen from the group consisting of H, OH, alkoxy and hydroxyalkoxy, where the alkoxy or hydroxyalkoxy groups may be branched or unbranched and may have 1-18 carbon atoms, and where Gly is chosen from the group of mono- and oligoglycoside radicals.

According to the invention, the flavonoids can however also be advantageously chosen from the group of substances having the generic structural formula

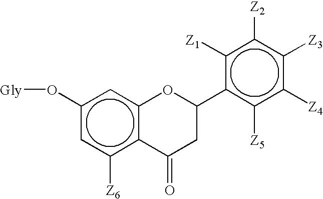

where Z₁-Z₆ independently of one another are chosen from the group consisting of H, OH, alkoxy and hydroxyalkoxy, where the alkoxy or hydroxyalkoxy groups can be branched or unbranched and may have 1-18 carbon atoms, and where Gly is chosen from the group consisting of mono- and oligoglycoside radicals.

Such structures may preferably be chosen from the group of substances of the generic structural formula

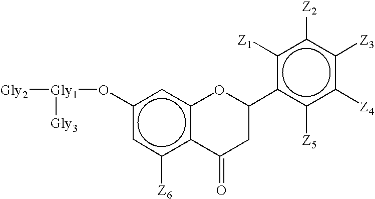

where Gly₁, Gly₂ and Gly₃ independently of one another are monoglycoside radicals. Gly₂ and Gly₃ may also, individually or together, represent saturations by hydrogen atoms.

Gly₁, Gly₂ and Gly₃ independently of one another are preferably chosen from the group consisting of hexosyl radicals, in particular rhamnosyl radicals and glucosyl radicals. However, if desired, it is also advantageous to use other hexosyl radicals, for example allosyl, altrosyl, galactosyl, gulosyl, idosyl, mannosyl and talosyl. It may also be advantageous according to the invention to use pentosyl radicals.

Z₁-Z₅ independently of one another are advantageously chosen from the group consisting of H, OH, methoxy, ethoxy and 2-hydroxyethoxy, and the flavone glycosides have the structure

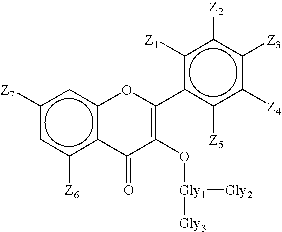

The flavone glycosides according to the invention are particularly advantageously chosen from the group represented by the following structure:

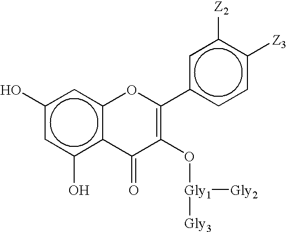

where Gly₁, Gly₂ and Gly₃ independently of one another are monoglycoside radicals. Gly₂ and Gly₃ can also, individually or together, represent saturations by hydrogen atoms.

Gly₁, Gly₂ and Gly₃ independently of one another are preferably chosen from the group consisting of hexosyl radicals, in particular rhamnosyl radicals and glucosyl radicals. However, if desired, it is also advantageous to use other hexosyl radicals, for example allosyl, altrosyl, galactosyl, gulosyl, idosyl, mannosyl and talosyl. It may also be advantageous according to the invention to use pentosyl radicals.

For the purposes of the present invention, it is particularly advantageous to choose the flavone glycoside(s) from the group consisting of α-glucosylrutin, α-glucosylmyricitrin, α-glucosylisoquercitrin and α-glucosylquercitrin.

One flavonoid which is particularly advantageous according to the invention is α-glucosylrutin. It is characterized by the following structure:

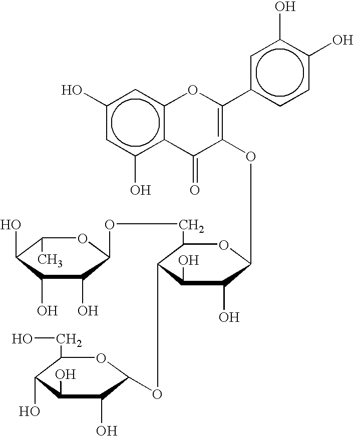

Another particularly advantageous flavonoid according to the invention is naringin (aurantiin, naringenine 7-rhamnoglucoside). It is characterized by the following structure:

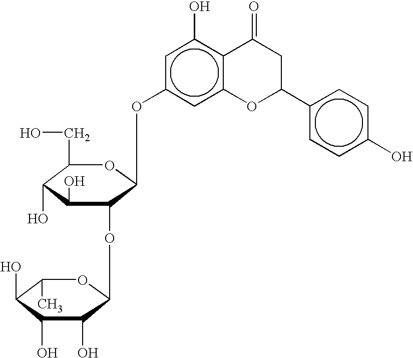

Another particularly advantageous flavonoid according to the invention is hesperidin (3′,5,7-trihydroxy-4′-methoxyflavanone-7-rutinoside, hesperidoside, hesperetin-7-O-rutinoside). It is characterized by the following structure:

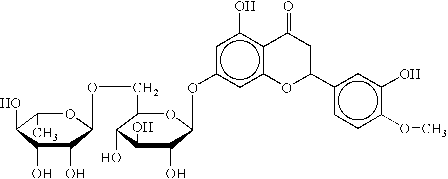

Another particularly advantageous flavonoid according to the invention is rutin (3,3′,4′,5,7-pentahydroxyflavone-3-rutinoside, quercetin-3-rutinoside, sophorin, Birutan, rutabion, taurutin, phytomelin, melin). It is characterized by the following structure:

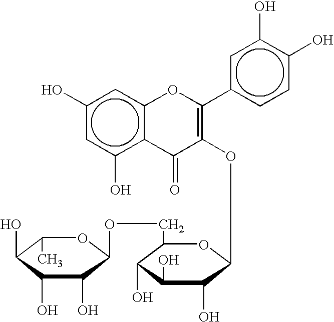

Another particularly advantageous flavonoid according to the invention is troxerutin (3,5-dihydroxy-3′,4′,7-tris(2-hydroxyethoxy)-flavone-3-(6-O-(6-deoxy-α-L-mannopyranosyl)-β-D-glucopyranoside)). It is characterized by the following structure:

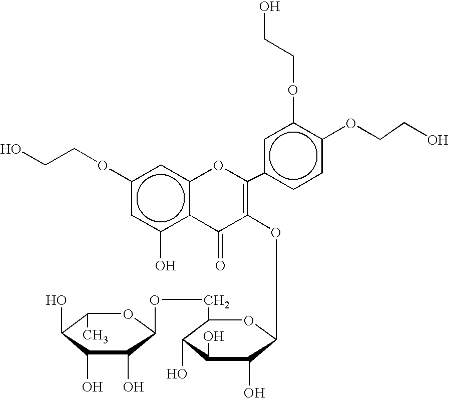

Another particularly advantageous flavonoid according to the invention is monoxerutin (3,3′,4′,5-tetrahydroxy-7-(2-hydroxyethoxy)flavone-3-(6-O-(6-deoxy-α-L-mannopyranosyl)-β-D-glucopyranoside)). It is characterized by the following structure:

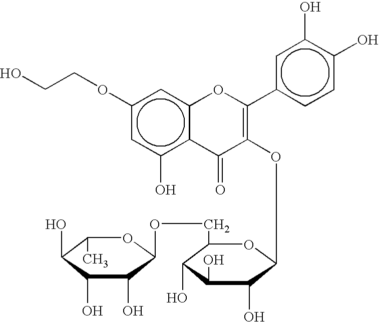

Another particularly advantageous flavonoid according to the invention is taxifolin (3,3′,4′,5,7-pentahydroxyflavanone). It is characterized by the following structure:

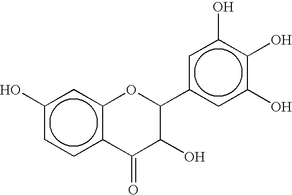

Another particularly advantageous flavonoid according to the invention is dihydrorobinetin (3,3′,4′,5′,7-pentahydroxyflavanone). It is characterized by the following structure:

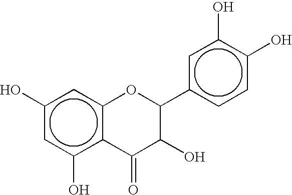

Another particularly advantageous flavonoid according to the invention is eriodictyol-7-glucoside (3′,4′,5,7-tetrahydroxyflavanbne-7-glucoside). It is characterized by the following structure:

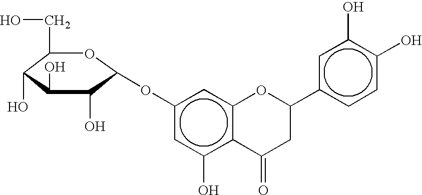

Another particularly advantageous flavonoid according to the invention is flavanomarein (3′,4′,7,8-tetrahydroxyflavanone-7-glucoside). It is characterized by the following structure:

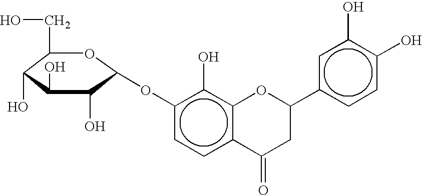

Another particularly advantageous flavonoid according to the invention is isoquercitrin (3,3′,4′,5,7-pentahydroxyflavanone-3-(β-D-glucopyranoside). It is characterized by the following structure:

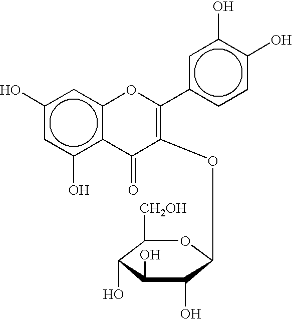

According to the invention, the flavone derivative(s) and/or flavanone derivative(s), in particular flavonoids, are advantageously present in cosmetic or dermatological preparations preferably in amounts of from 0.001% by weight to 10% by weight, preferably from 0.05% by weight to 5% by weight, particularly preferably 0.1-2.0% by weight, based on the total weight of the preparations.

According to the invention, the ascorbyl compound or the ascorbyl compounds, in particular vitamin C, is/are advantageously present in cosmetic or dermatological preparations preferably in amounts of from 0.001% by weight to 10% by weight, preferably from 0.05% by weight to 5% by weight, particularly preferably 0.1-2.05 by weight, based on the total weight of the preparations.

The novel combination of at least one flavone derivative and/or flavanone derivative, in particular at least one flavonoid and at least one ascorbyl compound, in particular vitamin C, is, for the purposes of this specification, also collectively referred to as “active ingredient according to the invention” or “active ingredient used according to the invention” or “active ingredient combination used according to the invention” or given synonymous designations.

The specification JP-A Hei-06-138,941 describes oral preparations containing water-soluble glucosides which can be chosen, for example, from the group consisting of α-glucosylrutin, α-glucosylmyricetin, α-glucosylisoquercitrin and α-glucosylquercitrin. The specification JP-A Hei-04-363,395 describes a method of preventing the decomposition of perfume constituents, which is characterized inter alia by an addition of α-glucosylrutin to the corresponding preparations. In addition, the specifications EP-A 586 303 and EP-A 595 694 describe the use of flavonoids as antioxidants or light protection substances in cosmetics.

However, these specifications do not contain any information which could point in the direction of the present invention.

It could therefore not have been foreseen by the person skilled in the art that the active ingredient combinations used according to the invention or cosmetic or dermatological preparations comprising them would

be more effective antioxidants

be more effective free-radical scavengers

better prevent the binding of harmful photoproducts to lipids, DNA and proteins

be more effective against skin ageing

better protect the skin against photoreactions

better prevent inflammatory reactions

than the active ingredients, active ingredient combinations and preparations of the prior art. In addition, it could not have been foreseen that the active ingredient combinations used according to the invention in cosmetic or dermatological preparations are more stable than the respective active ingredients used individually, something which applies in particular to the ascorbyl compounds and very particularly to vitamin C.

The invention therefore relates to the use of active ingredient combinations of flavones, flavanones or flavonoids and ascorbic acid and/or ascorbyl compounds as an antioxidant and also to its use for the treatment and/or prophylaxis of skin ageing caused as a result of oxidative stress, and of inflammatory reactions.

A particularly advantageous embodiment of the present invention is also the use of active ingredient combinations of flavones, flavanones or flavonoids and ascorbic acid and/or ascorbyl compounds for the treatment and/or prophylaxis of oxidative stress.

The cosmetic or dermatological preparations according to the invention can have the customary composition and can be used for the treatment, care and cleansing of the skin and/or the hair and as a make-up product in decorative cosmetics. They preferably comprise from 0.001% by weight to 10% by weight, preferably from 0.05% by weight to 5% by weight, in particular 0.1-2.0% by weight, based on the total weight of the preparations, of active ingredient combinations used according to the invention.

According to the invention, it is preferred to add such active ingredient combinations containing complexing agents to the active ingredient combinations or cosmetic or dermatological preparations used according to the invention.

Complexing agents are auxiliaries used in cosmetics or medicinal pharmaceutical technology which are known per se. By complexing undesired metals such as Mn, Fe, Cu and others, it is possible, for example, to prevent undesired chemical reactions in cosmetic or dermatological preparations.

Complexing agents, in particular chelating agents, form complexes with metal atoms. In the presence of one or more polybasic complexing agents, i.e. chelating agents, these complexes are metallacycles. Chelates are compounds in which a single ligand occupies more than one co-ordination site on a central atom. In this case, normally extended compounds are thus closed as a result of complex formation via a metal atom or a metal ion to form rings. The number of bonded ligands depends on the co-ordination number of the central metal. A prerequisite for formation of the chelate is that the compound reacting with the metal contains two or more atomic groupings which act as electron donors.

The complexing agent(s) can advantageously be chosen from the group of customary compounds, preferably at least one substance from the group consisting of tartaric acid and anions thereof, citric acid and anions thereof, aminopolycarboxylic acids and anions thereof (such as, for example, ethylenediaminetetraacetic acid (EDTA) and anions thereof, nitrilotriacetic acid (NTA) and anions thereof, hydroxyethylenediaminotriacetic acid (HOEDTA) and anions thereof, diethyleneaminopentaacetic acid (DPTA) and anions thereof, trans-1,2-diaminocyclohexanetetraacetic acid (CDTA) and anions thereof).

According to the invention, the complexing agent(s) is/are advantageously present in cosmetic or dermatological preparations preferably in amounts of from 0.01% by weight to 10% by weight, preferably from 0.05% by weight to 5% by weight, particularly preferably 0.1-2.0% by weight, based on the total weight of the preparations.

For use, according to the invention, the cosmetic and dermatological preparations are applied to the skin and/or the hair in an adequate amount in the customary manner for cosmetics.

Cosmetic and dermatological preparations according to the invention can be in various forms. Thus, they can, for example, be a solution, a water-free preparation, an emulsion or microemulsion of the water-in-oil (W/O) type or of the oil-in-water (O/W) type, a multiple emulsion, for example of the water-in-oil-in-water (W/O/W) type, a gel, a solid stick, an ointment or else an aerosol. It is also advantageous to administer isoquercitrin in capsule form, for example in collagen matrices and other customary encapsulation materials, for example as cellulose capsules, in gelatin, wax matrices or liposomally encapsulated. In particular, wax matrices, as are described in DE-A 43 08 282, have proven to be favourable.

For the purposes of the present invention, it is also possible and advantageous to incorporate active ingredient combinations used according to the invention into aqueous systems or surfactant preparations for cleansing the skin and the hair.

The cosmetic and dermatological preparations according to the invention, can comprise cosmetic auxiliaries such as are usually used in such preparations, for example preservatives, bactericides, perfumes, antifoams, dyes, pigments which have a colouring action, thickeners, surfactants, emulsifiers, softeners, moisturizers and/or humectants, fats, oils, waxes or other customary constituents of a cosmetic or dermatological preparation, such as alcohols, polyols, polymers, foam stabilizers, electrolytes, organic solvents or silicone derivatives.

In particular, active ingredient combinations used according to the invention can also be combined with other antioxidants and/or free-radical scavengers.

Such antioxidants are advantageously chosen from the group consisting of amino acids (for example glycine, histidine, tyrosine, tryptophan) and derivatives thereof, imidazoles (for example urocanic acid) and derivatives thereof, peptides such as D,L-carnosine, D-carnosine, L-carnosine and derivatives thereof (for example anserine), carotenoids, carotenes (for example α-carotene, β-carotene, lycopene) and derivatives thereof, chlorogenic acid and derivatives thereof, lipoic acid and derivatives thereof (for example dihydrolipoic acid), aurothioglucose, propylthiouracil and other thiols (for example thioredoxin, glutathione, cysteine, cystine, cystamine and the glycosyl, N-acetyl, methyl, ethyl, propyl, amyl, butyl and lauryl, palmitoyl, oleyl, γ-linoleyl, cholesteryl and glyceryl esters thereof) and salts thereof, dilauryl thiodipropionate, distearyl thiodipropionate, thiodipropionic acid and derivatives thereof (esters, ethers, peptides, lipids, nucleotides, nucleosides and salts) and sulphoximine compounds (for example buthionine sulphoximines, homocysteine sulphoximine, buthionine sulphones, penta-, hexa- and heptathionine sulphoximine) in very low tolerated doses (for example pmol to μmol/kg), and furthermore (metal) chelating agents (for example α-hydroxy-fatty acids, palmitic acid, phytic acid, lactoferrin), α-hydroxy acids (for example citric acid, lactic acid, malic acid), humic acid, bile acid, bile extracts, bilirubin, biliverdin, EDTA, EGTA and derivatives thereof, unsaturated fatty acids and derivatives thereof (for example γ-linolenic acid, linoleic acid, oleic acid), folic acid and derivatives thereof, ubiquinone and ubiquinol and derivatives thereof, tocopherols and derivatives (for example vitamin E acetate), vitamin A and derivatives (vitamin A palmitate) and coniferyl benzoate of benzoic resin, rutic acid and derivatives thereof, butylated hydroxytoluene, butylated hydroxyanisole, nordihydrbguaiac resin acid, nordihydroguaiaretic acid, trihydroxybutyrophenone, uric acid and derivatives thereof, mannose and derivatives thereof, sesamol, sesamolin, zinc and derivatives thereof (for example ZnO, ZnSO₄), selenium and derivatives thereof (for example selenium methionine), stilbenes and derivatives thereof (for example stilbene oxide, trans-stilbene oxide) and the derivatives of these active ingredients mentioned which are suitable according to the invention (salts, esters, ethers, sugars, nucleotides, nucleosides, peptides and lipids).

The amount of the abovementioned antioxidants (one or more compounds) in the preparations is preferably from 0.001 to 30% by weight, particularly preferably 0.05-20% by weight, in particular 1-10% by weight, based on the total weight of the preparation.

If vitamin E and/or derivatives thereof is or are the antioxidant or antioxidants, it is advantageous to choose the respective concentrations thereof from the range 0.001-10% by weight, based on the total weight of the preparation.

If vitamin A or vitamin A derivatives or carotenes or derivatives thereof is or are the antioxidant or antioxidants, it is advantageous to choose the respective concentrations thereof from the range 0.001-10% by weight, based on the total weight of the preparation.

Emulsions according to the invention are advantageous and comprise, for example, said fats, oils, waxes and other fatty substances, and also water and an emulsifier, as is customarily used for this type of formulation.

The lipid phase can advantageously be chosen from the following group of substances:

mineral oils, mineral waxes;

oils, such as triglycerides of capric or of caprylic acid, also natural oils such as, for example, castor oil;

fats, waxes and other natural and synthetic fatty substances, preferably esters of fatty acids with alcohols of low C number, for example with isopropanol, propylene glycol or glycerol, or esters of fatty alcohols with alkanoic acids of low C number or with fatty acids;

alkyl benzoates;

silicone oils, such as dimethylpolysiloxanes, diethylpolysiloxanes, diphenylpolysiloxanes and mixed forms thereof.

For the purposes of the present invention, the oil phase of the emulsions, oleogels and hydrodispersions or lipodispersions is advantageously selected from the group consisting of esters of saturated and/or unsaturated, branched and/or unbranched alkanecarboxylic acids having a chain length of from 3 to 30 carbon atoms and saturated and/or unsaturated, branched and/or unbranched alcohols having a chain length of from 3 to 30 carbon atoms, from the group consisting of esters of aromatic carboxylic acids and saturated and/or unsaturated, branched and/or unbranched alcohols having a chain length of from 3 to 30 carbon atoms. Such ester oils can then be advantageously chosen from the group consisting of isopropyl myristate, isopropyl palmitate, isopropyl stearate, isopropyl oleate, n-butyl stearate, n-hexyl laurate, n-decyl oleate, isooctyl stearate, isononyl stearate, isononyl isononanoate, 2-ethylhexyl palmitate, 2-ethylhexyl laurate, 2-hexyldecyl stearate, 2-octyldodecyl palmitate, oleyl oleate, oleyl erucate, erucyl oleate, erucyl erucate and synthetic, semi-synthetic and natural mixtures of such esters e.g. jojoba oil.

The oil phase can also advantageously be selected from the group consisting of branched and unbranched hydrocarbons and hydrocarbon waxes, silicone oils, dialkyl ethers, from the group consisting of saturated or unsaturated, branched or unbranched alcohols, and also fatty acid triglycerides, namely the triglycerol ester of saturated and/or unsaturated, branched and/or unbranched alkanecarboxylic acids having a chain length of from 8 to 24, in particular 12-18, carbon atoms. The fatty acid triglycerides can advantageously be selected, for example, from the group consisting of synthetic, semi-synthetic and natural oils, e.g. olive oil, sunflower oil, soybean oil, peanut oil, rapeseed oil, almond oil, palm oil, coconut oil, palm kernel oil and the like.

For the purposes of the present invention, any mixtures of such oil and wax components can also advantageously be used. When required, it may also be advantageous to use waxes, for example cetyl palmitate, as the sole lipid component of the oil phase.

The oil phase is advantageously chosen from the group consisting of 2-ethylhexyl isostearate, octyldodecanol, isotridecyl isononanoate, isoeicosane, 2-ethylhexyl cocoate, C₁₂-C₁₅-alkyl benzoate, caprylic/capric triglyceride and dicaprylyl ether.

Mixtures of C₁₂-C₁₅-alkyl benzoate and 2-ethylhexyl isostearate, mixtures of C₁₂-C₁₅-alkyl benzoate and isotridecyl isononanoate and mixtures of C₁₂-C₁₅-alkyl benzoate, 2-ethylhexyl isostearate and isotridecyl isononanoate are particularly advantageous.

For the purposes of the present invention, of the hydrocarbons, paraffin oil, squalane and squalene can advantageously be used.

The oil phase can advantageously also contain cyclic or linear silicone oils or can consist entirely of such oils, although it is preferable to use an additional content of other oil phase components in addition to the silicone oil or silicone oils.

Cyclomethicone (octamethylcyclotetrasiloxane) is advantageously used as the silicone oil to be used according to the invention. However, other silicone oils can also be advantageously used for the purposes of the present invention, for example hexamethylcyclotrisiloxane, polydimethylsiloxane, poly(methylphenylsiloxane).

Mixtures of cyclomethicone and isotridecyl isononanoate and mixtures of cyclomethicone and 2-ethylhexyl isostearate are particularly advantageous.

If appropriate, the aqueous phase of the preparations according to the invention advantageously comprises alcohols, diols or polyols of low C number and ethers thereof, preferably ethanol, isopropanol, propylene glycol, glycerol, ethylene glycol, ethylene glycol monoethyl or monobutyl ether, propylene glycol monomethyl, monoethyl or monobutyl ether, diethylene glycol monomethyl or monoethyl ether and analogous products, also alcohols of low C number, for example ethanol, isopropanol, 1,2-propanediol and glycerol, and, in particular, one or more thickeners, which can advantageously b chosen from the group consisting of silicon dioxide, aluminium silicates, polysaccharides and derivatives thereof, for example hyaluronic acid, xanthan gum and hydroxypropylmethylcellulose, particularly advantageously from the group consisting of polyacrylates, preferably a polyacrylate from the group consisting of Carbopols, for example Carbopols of types 980, 981, 1382, 2984 and 5984, in each case individually or in combination.

In particular, mixtures of the abovementioned solvents are used. In the case of alcoholic solvents, water may be a further constituent.

Emulsions according to the invention are advantageous and comprise, for example, said fats, oils, waxes and other fatty substances, and also water and an emulsifier, as is customarily used for this type of formulation.

Gels according to the invention customarily comprise alcohols of low C number, for example ethanol, isopropanol, 1,2-propanediol, glycerol, and water and/or an abovementioned oil in the presence of a thickener which, in the case of oily-alcoholic gels, is preferably silicon dioxide or an aluminium silicate, and in the case of aqueous-alcoholic or alcoholic gels, is preferably a polyacrylate.

Suitable propellants for preparations according to the invention which can be sprayed from aerosol containers are the customary known readily volatile, liquefied propellants, for example hydrocarbons (propane, butane, isobutane), which may be used alone or in mixtures with one another. Compressed air can also be used advantageously.

Preparations according to the invention can advantageously also comprise substances which absorb UV radiation in the UVB region, the total amount of filter substances being, for example, from 0.1% by weight to 30% by weight, preferably from 0.5 to 10% by weight, in particular from 1.0 to 6.0% by weight, based on the total weight of the preparations, in order to provide cosmetic formulations which protect the skin or hair from the entire range of ultraviolet radiation. They can also be used as sunscreen compositions for hair or skin.

If the preparations according to the invention comprise UVB filter substances, these may be oil-soluble or water-soluble. Advantageous oil-soluble UVB filter substances are, for example:

3-benzylidenecamphor derivatives, preferably 3-(4-methylbenzylidene)camphor and 3-benzylidenecamphor;

4-aminobenzoic acid derivatives, preferably 2-ethylhexyl 4-(dimethylamino)benzoate and amyl 4-(dimethylamino)benzoate;

esters of cinnamic acid, preferably 2-ethylhexyl 4-methoxycinnamate and isopentyl 4-methoxycinnamate;

esters of salicylic acid, preferably 2-ethylhexyl salicylate, 4-isopropylbenzyl salicylate and homomenthyl salicylate,

derivatives of benzophenone, preferably 2-hydroxy-4-methoxybenzophenone, 2-hydroxy-4-methoxy-4′-methylbenzophenone and 2,2′-dihydroxy-4-methoxybenzophenone;

esters of benzalmalonic acid, preferably di(2-ethylhexyl) 4-methoxybenzalmalonate and -2,4,6-tris(p-2-ethylhexoxy carbonyl-anilino)-1,3,5-triazine.

Advantageous water-soluble UVB filters are, for example:

salts of 2-phenylbenzimidazole-5-sulphonic acid, such as its sodium, potassium or its triethanolammonium salt, and the sulphonic acid itself;

sulphonic acid derivatives of benzophenones, preferably 2-hydroxy-4-methoxybenzophenone-5-sulphonic acid and its salts;

sulphonic acid derivatives of 3-benzylidenecamphor, such as, for example 4-(2-oxo-3-bornylidenemethyl)benzenesulphonic acid, 2-methyl-5-(2-oxo-3-bornylidenemethyl)sulphonic acid and their salts, and also 1,4-di(2-oxo-10-sulpho-3-bornylidenemethyl)benzene and its salts (the corresponding 10-sulphato compounds, for example the corresponding sodium, potassium or triethanolammonium salt) also referred to as benzene-1,4-di(2-oxo-3-bornylidenemethyl)-10-sulphonic acid.

The list of the UVB filters mentioned, which can be used in combination with the active ingredient combinations according to the invention, is not of course intended to be limiting.

The invention also relates to the use of a combination of the active ingredient combinations used according to the invention with at least one UVB filter as an antioxidant and to the use of a combination of the active ingredient combinations used according to the invention with at least one UVB filter as an antioxidant in a cosmetic or dermatological preparation.

It can also be advantageous to combine the active ingredient combinations used according to the invention with UVA filters, which have to date customarily been present in cosmetic preparations. These substances are preferably derivatives of dibenzoylmethane, in particular 1-(4′-tert-butylphenyl)-3-(4′-methoxyphenyl)propane-1,3-dione and 1-phenyl-3-(4′-isopropylphenyl)propane-1,3-dione. These combinations and preparations comprising these combinations are also provided by the invention. The amounts used are as for the UVB combination.

The invention also relates to the use of a combination of active ingredient combinations used according to the invention with at least one UVA filter as an antioxidant and to the use of a combination of the active ingredient combinations according to the invention with at least one UVA filter as an antioxidant in a cosmetic or dermatological preparation.

The invention also relates to the use of a combination of active ingredient combinations used according to the invention with at least one UVA filter and at least one UVB filter as an antioxidant and to the use of a combination of isoquercitrin with at least one UVA filter and at least one UVB filter as an antioxidant in a cosmetic or dermatological preparation.

Cosmetic and dermatological preparations having an effective content of combinations of active ingredients according to the invention can also contain inorganic pigments which are normally used in cosmetics for protecting the skin against UV rays. These are oxides of titanium, zinc, zirconium, silicon, manganese, cerium and mixtures thereof, and modifications in which the oxides are the active agents. Particular preference is given to pigments based on titanium dioxide.

These combinations of UVA filter and pigment and preparations which comprise this combination are also provided by the invention. The quantities used may be as stated for the aforementioned combinations.

The cosmetic and dermatological preparations for protecting the hair against UV rays according to the invention are, for example, shampoos, preparations which are applied to the hair when rinsing the hair before or after shampooing, before or after permanent waving, before or after colouring or bleaching, preparations for blow-drying or setting the hair, preparations for colouring or bleaching, a styling and treatment lotion, a hairspray or a permanent wave solution.

The cosmetic and dermatological preparations comprise active ingredients and auxiliaries which are usually used for this type of preparation for hair care and hair treatment. Auxiliaries include preservatives, surfactants, antifoams, thickeners, emulsifiers, fats, oils, waxes, organic solvents, bactericides, perfumes, dyes or pigments whose task is to colour the hair or the cosmetic or dermatological preparation itself, electrolytes and anti-grease substances.

For the purposes of the present invention, electrolytes are taken to mean water-soluble alkali metal, ammonium, alkaline earth metal (including magnesium) and zinc salts of inorganic anions and any mixtures of such salts, it being necessary to ensure that these salts are pharmaceutically or cosmetically safe.

The anions according to the invention are preferably selected from the group consisting of chlorides, sulphates and hydrogensulphates, phosphates, hydrogenphosphates and linear and cyclic oligophosphates and carbonates and hydrogencarbonates.

Cosmetic preparations which are in the form of a skin cleanser or shampoo preferably comprise at least one anionic, nonionic or amphoteric surfactant, or also mixtures of such substances, the active ingredient combinations according to the invention in aqueous medium, and auxiliaries usually used for this purpose. The surfactant and the mixtures of these substances can be present in the shampoo in a concentration of between 1% by weight and 50% by weight.

If the cosmetic or dermatological preparations are in the form of a lotion which is rinsed out and applied, for example, before or after bleaching, before or after shampooing, between two shampooing steps, before or after permanent waving, they are for example, aqueous or aqueous-alcoholic solutions optionally comprising surfactants in a concentration of between 0.1 and 10% by weight, preferably between 0.2 and 5% by weight.

These cosmetic or dermatological preparations can also be in the form of aerosols with the auxiliaries usually used for this purpose.

A cosmetic preparation in the form of a lotion which is not rinsed out, in particular a lotion for setting the hair, a lotion which is used for blow drying the hair, a styling and treatment lotion, is generally in the form of an aqueous, alcoholic or aqueous-alcoholic solution, and contains at least one cationic, anionic, nonionic or amphoteric polymer or also mixtures thereof, and also active ingredient combinations used according to the invention in an effective concentration. The amount of polymers used is, for example, between 0.1 and 10% by weight, preferably between 0.1 and 3% by weight.

Cosmetic preparations for treating and caring for the hair which contain active ingredients according to the invention can be in the form of emulsions which are of the nonionic or anionic type. Nonionic emulsions comprise, in addition to water, oils or fatty alcohols which may, for example, also be polyethoxylated or polypropoxylated, or also mixtures of the two organic components. These emulsions optionally contain cationic surfactants.

According to the invention, cosmetic preparations for treating and caring for the hair can be in the form of gels which, in addition to an effective content of isoquercitrin and solvents usually used therefor, preferably water, also contain organic thickeners, e.g. gum arabic, xanthan gum, sodium alginate, cellulose derivatives, preferably methylcellulose, hydroxymethylcellulose, hydroxyethylcellulose, hydroxypropylcellulose, hydroxypropylmethylcellulose or inorganic thickeners, for example aluminium silicates such as, for example, bentonites, or a mixture of polyethylene glycol and polyethylene glycol stearate or distearate. The thickener is present in the gel, for example, in an amount between 0.1 and 30% by weight, preferably between 0.5 and 15% by weight.

The amount of isoquercitrin in a product intended for hair is preferably from 0.05% by weight to 10% by weight, in particular from 0.5% by weight to % by weight, based on the total weight of the product.

Aqueous cosmetic cleansers according to the invention or low-water or water-free cleanser concentrates intended for aqueous cleansing may contain anionic, nonionic and/or amphoteric surfactants, for example

conventional soaps, e.g. fatty acid salts of sodium

alkyl sulphates, alkyl ether sulphates, alkanesulphonates and alkylbenzenesulphonates

sulphoacetates

sulphobetaines

sarcosinates

amidosulphobetaines

sulphosuccinates

sulphosuccinic acid monoesters

alkyl ether carboxylates

protein-fatty acid condensates

alkylbetaines and amidobetaines

fatty acid alkanolamides

polyglycol ether derivatives

Cosmetic preparations which are cosmetic skin cleansing preparations can be in liquid or solid form. In addition to active ingredient combinations according to the invention, they preferably contain at least one anionic, nonionic or amphoteric surfactant or mixtures thereof, if desired one or more electrolytes and auxiliaries usually used for this purpose. The surfactant can be present in the cleansing preparations in a concentration of between 1 and 94% by weight, based on the total weight of the preparations.

Cosmetic preparations which are a shampoo preferably comprise, in addition to an effective amount of isoquercitrin, at least one anionic, nonionic or amphoteric surfactant or mixtures thereof, if desired one electrolyte according to the invention and auxiliaries which are usually used for this purpose. The surfactant can be present in the shampoo in a concentration of between 1% by weight and 94% by weight.

The compositions according to the invention comprise, in addition to the aforementioned surfactants, water and, when required, the additives usual in cosmetics, for example perfume, thickeners, dyes, deodorants, antimicrobial substances, refatting agents, complexing agents and sequestering agents, pearlizing agents, plant extracts, vitamins, active ingredients and the like.

The present invention also relates to a cosmetic method of protecting the skin and hair against oxidative or photooxidative processes which is characterized in that a cosmetic composition which comprises: an effective concentration of active ingredient combinations used according to the invention is applied in a sufficient quantity to the skin or hair.

The present invention likewise also covers a method of protecting cosmetic or dermatological preparations against oxidation or photooxidation, these preparations being, for example, preparations for the treatment and care of hair, in particular hair colorants, hairsprays, shampoos, colour shampoos, and also make-up products such as, for example, nail varnishes, lipsticks, foundations, washing and shower preparations, creams for the treatment or care of skin or all other cosmetic preparations whose constituents may be associated with stability problems because of oxidation or photooxidation during storage, characterized in that the cosmetic preparations have an effective content of isoquercitrin.

The amount of active ingredient compositions used according to the invention in these preparations is preferably 0.01-10% by weight, preferably 0.05-5% by weight, in particular 0.1-2.0% by weight, based on the total weight of the preparations.

The invention also relates to the process for the preparation of the cosmetics compositions according to the invention, which is characterized in that active ingredient combinations according to the invention are incorporated into cosmetic or dermatological formulations in a manner known per se.

Although the cosmetic or dermatological preparations achieved according to the invention and the active ingredient combinations present therein are notable for increased stability towards an oxidative effect, storage forms which are preferable are those in which the entry of atmospheric oxygen is reduced. Thus, filling under an inert gas, in particular nitrogen, is, for example, advantageous. A particularly advantageous packaging has proven to be aluminium tubes.

The examples below serve to illustrate the present invention without limiting it. Unless stated otherwise, all quantities, proportions and percentages are by weight and based on the total amount or on the total amount of the preparations.

### EXAMPLE 1

O/W cream

| | % by wt. |
|---|---|
| Glyceryl stearate | 5.00 |
| Cetyl alcohol | 5.00 |
| Isopropyl palmitate | 7.00 |
| Cyclomethicone | 5.00 |
| Ascorbic acid | 3.00 |
| α-Glucosylrutin | 0.30 |
| NaOH, 45% strength | 1.00 |
| Butylene glycol | 3.00 |
| Na₂H₂EDTA | 0.20 |
| Dyes, perfume, preservatives | q.s. |
| Water | ad 100.00 |

### EXAMPLE 2

O/W lotion

| | % by wt. |
|---|---|
| Steareth-20 | 3.00 |
| Cetyl alcohol | 3.00 |
| Cyclomethicone | 6.00 |
| Carbomer | 0.60 |
| Na₂H₂EDTA | 0.20 |
| Butylene glycol | 3.00 |
| NaOH, 45% strength | 0.40 |
| Ascorbic acid | 0.50 |
| α-Glucosylrutin | 0.10 |
| Dyes, perfume, preservatives | q.s. |
| Water | ad 100.00 |

### EXAMPLE 3

W/O cream

| | % by wt. |
|---|---|
| Polyglyceryl-2-dipolyhydroxystearate | 5.00 |
| Caprylic/capric triglycerides | 15.00 |
| Butylene glycol | 3.00 |
| Na₂H₂EDTA | 0.20 |
| MgSO₄ | 0.70 |
| NaOH, 45% strength | 0.32 |
| Ascorbic acid | 1.00 |
| α-Glucosylrutin | 0.20 |
| Dyes, perfume, preservatives | q.s. |
| Water | ad 100.00 |

### EXAMPLE 4

O/W gel

| | % by wt. |
|---|---|
| Xanthan gum | 2.00 |
| Butylene glycol | 3.00 |
| Na₂H₂EDTA | 0.20 |
| NaOH, 45% strength | 0.32 |
| Ascorbic acid | 1.00 |
| α-Glucosylrutin | 0.20 |
| Dyes, perfume, preservatives | q.s. |
| Water | ad 100.00 |

### EXAMPLE 5

O/W hair treatment

| | % by wt. |
|---|---|
| Bis-diglyceryl polyacyladipate-2 | 3.00 |
| Behenyl alcohol | 4.00 |
| Butylene glycol | 3.00 |
| Cetrimonium chloride | 5.00 |
| Citric acid | 0.50 |
| Na₂H₂EDTA | 0.20 |
| NaOH, 45% strength | 0.16 |
| Ascorbic acid | 0.50 |
| α-Glucosylrutin | 0.10 |
| Dyes, perfume, preservatives | q.s. |
| Water | ad 100.00 |

1. Use of at least one active ingredient chosen from the group consisting of flavones, flavanones and flavonoids for protecting at least one active ingredient chosen from the group consisting of ascorbic acid and ascorbyl compounds from oxidation.

2. Use according to claim 1, characterized in that the active ingredient(s) chosen from the group consisting of flavones, flavanones and flavonoids is/are present in cosmetic or dermatological preparations in an effective amount.

3. Use according to claim 2, characterized in that the active ingredient(s) chosen from the group consisting of flavones, flavanones and flavonoids is/are present in cosmetic or topical dermatological preparations in concentrations of 0.01-10% by weight, preferably 0.05-5% by weight, in particular 0.1-2.0% by weight, based on the total weight of the preparations.

4. Use according to claim 1, characterized in that the active ingredient(s) chosen from the group consisting of ascorbic acid and ascorbyl compounds is present in cosmetic or dermatological preparations in an effective amount.

5. Use according to claim 4, characterized in that the active ingredient(s) chosen from the group consisting of ascorbic acid and ascorbyl compounds is/are present in cosmetic or topical dermatological preparations in concentrations of 0.001-10% by weight, preferably 0.05-5% by weight, in particular 0.1-2.0% by weight, based on the total weight of the preparation.

6. Use-according to claim 1, characterized in that the active ingredient chosen from the group consisting of flavones, flavanones and flavonoids is α-glucosylrutin.

7. Use according to claim 2, characterized in that the cosmetic or dermatological preparations additionally comprise one or more complexing agents.

8. Use according to claim 7, characterized in that the complexing agent(s) is/are chosen from the group consisting of tartaric acid and anions thereof, citric acid and anions thereof, aminopolycarboxylic acids and anions thereof (such as, for example, ethylenediaminetetraacetic acid and anions thereof, nitrilotriacetic acid and anions thereof, hydroxyethylenediaminotriacetic acid and anions thereof, diethyleneaminopentaacetic acid and anions thereof, and trans-1,2-diaminocyclohexanetetraacetic acid and anions thereof).

9. Use according to claim 7, characterized in that the complexing agent(s) is/are present in the cosmetic or dermatological preparations preferably in amounts from 0.01% by weight to 10% by weight, preferably from 0.05% by weight to 5% by weight, particularly preferably 0.1-2.0% by weight, based on the total weight of the preparations.
